(19) United States
(12) Patent Application Publication
(10) Pub. No.: US 2021/0327584 A1
(43) Pub. Date: Oct. 21, 2021

(54) DECISION SUPPORT SOFTWARE SYSTEM FOR SLEEP DISORDER IDENTIFICATION

(71) Applicant: KONINKLIJKE PHILIPS N.V., Eindhoven (NL)

(72) Inventors: Andrew VAKULIN, Eindhoven (NL); Peter Guthrie CATCHESIDE, Eindhoven (NL); Nicole LOVATO, Eindhoven (NL); Ronald Douglas MCEVOY, Eindhoven (NL); Angus Keith WALLACE, Eindhoven (NL); Karen Jane SMALL, Eindhoven (NL); Bryn JEFFRIES, Eindhoven (NL); Fabio Tozeto RAMOS, Eindhoven (NL); Christopher GORDON, Eindhoven (NL); Tracey Leanne SLETTEN, Eindhoven (NL); Leon Colburn LACK, Eindhoven (NL); Greg Nelson GARCIA MOLINA, Eindhoven (NL); Yash Parag MOKASHI, Pittsburgh, PA (US); Jesse SALAZAR, Gibsonia, PA (US); Monica H BUSH, Murrysville, PA (US); Kousalya RONDINELLI, Pittsburgh, PA (US); Jenna M. SCHABDACH, Eindhoven (NL); Craig OAKS, Pittsburgh, PA (US)

(21) Appl. No.: 17/287,368

(22) PCT Filed: Oct. 22, 2019
(86) PCT No.: PCT/AU2019/051147

§ 371 Date: Apr. 21, 2021

Related U.S. Application Data
(60) Provisional application No. 62/812,066, filed on Feb. 28, 2019.

(30) Foreign Application Priority Data
Oct. 22, 2018 (AU) 2018904007
Jul. 29, 2019 (AU) 2019902689

(51) Int. Cl. G16H 50/20; G16H 50/70; G16H 40/67; G16H 10/20; G06F 40/40; A61B 5/0205; A61B 5/021; A61B 5/08; A61B 5/11; A61B 5/113; A61B 5/024; A61B 7/00

(52) U.S. Cl. CPC G16H 50/20; G16H 50/70; G16H 40/67; G16H 10/20; G06F 40/40; A61B 5/0205; G16H 50/30; A61B 5/0816; A61B 5/1118; A61B 5/113; A61B 5/024; A61B 7/003; A61B 5/021

(57) ABSTRACT

A method for cluster-based recommendation generation regarding sleep disorders. A server system transmitting query program code to a client device, wherein the query program code is executable by the client device to transmit one or more response objects encoding a response and further responses to the server system. The server system receiving the one or more response objects from the client device and determining the response and further responses encoded in the response objects. A clustering module of the server system identifying one or more clusters of sleep disorder user data that most closely relate to the determined responses. A recommendation module of the server system identifying a sleep disorder based on the determined responses and clusters. The recommendation module generating one or more recommendations based on the identified sleep disorder, the determined responses and the identified clusters. The server system encoding the generated one or more recommendations in a recommendations object and making it accessible to the client device.

### TECHNICAL FIELD

The present disclosure is generally directed to decision support software systems and methods for sleep disorder identification.

### BACKGROUND

Many people suffer from difficulties in getting to sleep, or staying asleep, according to an appropriate sleep cycle.

Individuals with untreated sleep disorders are understood to present higher risks of physical health issues such as high blood pressure, heart disease, diabetes, stroke, obesity, and reduced immunity. In addition, sleep disorders have been linked to mental health issues such as depression and anxiety.

Sleep disorders also naturally lead to fatigue, which is widely understood to negatively impact alertness, cognitive function and the ability to remain awake. Fatigue is consequently identified as a major contributor to occupational accident risk, especially for the more than 1.4 million Australians or 15 million Americans who engage in shift work. On average, at least one Australian will die each day from falling asleep at the wheel of a motor vehicle due to insufficient sleep.

The economic costs of sleep disorders and inadequate sleep are also substantial. It is estimated that the costs of inadequate sleep during the 2016/2017 financial year in Australia alone equate to more than AUD 66 billion, including costs to the healthcare system, productivity losses, and wellbeing costs.

There are many causes of sleep difficulties. However, five of the most common sleep disorders are: insomnia, snoring/obstructive sleep apnoea, delayed sleep wake phase disorder, chronic sleep restriction, and shift work disorder.

Insomnia is a sleep disorder in which people have difficulty falling or staying asleep. Between ten and thirty percent of adults have insomnia at any given point in time and up to fifty percent of adults will suffer insomnia in a given year. Of those, about six percent of people will suffer insomnia which is not due to another underlying problem and which lasts for longer than a month. Insomnia may broadly take two forms: sleep onset insomnia, and sleep maintenance insomnia. Sleep onset insomnia describes difficulty falling asleep at bed time, while sleep maintenance insomnia involves frequent and/or extended nocturnal awakenings that total more than 30 minutes of wakefulness across the night. Both forms are largely precipitated by maladaptive learning and conditioning processes and, particularly in the case of sleep onset insomnia, misalignment of circadian rhythms.

Obstructive sleep apnoea is caused by complete or partial obstructions of the upper airway and is characterised by repetitive episodes of shallow or paused breathing during sleep. Obstructive sleep apnoea commonly causes excessive daytime sleepiness, and may case sufferers to fall asleep for brief periods during daytime activities. Obstructive sleep apnoea is commonly accompanied by snoring, which is known to cause sleep deprivation to sufferers and those around them.

Delayed sleep phase disorder is a chronic dysregulation of a person's circadian rhythm, and will affect the timing of sleep, peak periods of alertness, the core body temperature rhythm, and hormonal and other daily cycles. It is thought that those suffering delayed sleep phase disorder may have a circadian rhythm which is significantly longer than 24 hours. As a result, the effect of delayed sleep phase disorder on those attempting to follow a 24 hour schedule has been compared to constant jet lag.

Chronic sleep restriction, also known as sleep deprivation, is the persistent condition of not having enough sleep. Aside from fatigue, the physiological effects of sleep restriction may include: confusion, memory lapses, depression, headaches, increased blood pressure, increased risk of diabetes, and lowered immunity.

Shift work disorder provides similar symptoms to insomnia or excessive sleepiness and occurs as a result of transient work schedules. Shift work requires workers to attempt sleep at biologically inappropriate times of the day. Those suffering shift work disorder are unable to obtain at these times. While a typical shift worker may for example obtain 6.5 hours of sleep in a 24 hour period, those suffering from shift work disorder will report obtaining far less sleep.

With the relative widespread availability of sensor devices for tracking health data, such as smart watch based fitness trackers and smartphones, a large amount of relevant data can be available to assist in identification of sleep disorders and assess recommendations to address identified sleep disorders. As the sensor devices may be used for prolonged periods by users, a significant amount of relevant information may be available for analysis. Further, as information from greater number of users of sensor devices is available, there are opportunities to correlate data across a large number of users to better identify sleep disorders and identify more effective recommendations.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

Some embodiments relate to a computer-implemented method for cluster-based recommendation generation regarding sleep disorders, the method comprising:

a server system transmitting query program code to a client device, wherein the query program code is executable by the client device to cause the client device to transmit one or more response objects encoding a response and further responses to the server system;

the server system receiving the one or more response objects from the client device and determining the response and further responses encoded in the one or more response objects;

a clustering module of the server system identifying one or more clusters of sleep disorder user data that most closely relate to the determined response and determined further responses;

a recommendation module of the server system identifying a sleep disorder based on the determined response, the determined further responses and the identified one or more clusters;

the recommendation module generating one or more recommendations based on the identified sleep disorder, the determined response, the determined further responses and the identified one or more clusters; and the server system encoding the generated one or more recommendations in a recommendations object and making the recommendations object accessible to the client device.

The query program code may comprise a query object and one or more further query objects; the response may be a response to the query object; the further responses may be responses to a subset of the one or more further query objects; and the subset of the one or more further query objects may be determined based on the response object.

The determining of the subset of the one or more further query objects may be performed by the server system. Alternatively, the determining of the subset of the one or more further query objects is performed by the client device.

At least one of the one or more further query objects may be directed to a sensor and at least one of the one or more response objects comprises health data from the sensor. The health data may comprise one or more of: cardiac activity measurement data, physical activity measurement data, blood pressure measurement data, respiratory activity measurement data, heart rate data, movement data, breathing sound data or breathing rate data.

The recommendation module may further identify one or more secondary sleep disorders based on the determined response, the determined further responses and the identified one or more clusters.

The clustering module may perform clustering based on any one of: similarity learning, distance metrics, feature vector comparison or agglomerative clustering.

At least one of the one or more response objects may comprise free text and a natural language processing module of the server system is configured to process the free text to determine an input to the recommendation module.

The method may further comprise:

receiving feedback input via the client device regarding the one or more recommendations;

reconfiguring a recommendation model of the recommendation module to take into account the received feedback input.

Some embodiments relate to a computer-implemented method for cluster-based sleep disorder related recommendation prioritization, the method comprising:

a server system transmitting query program code to a client device, wherein the query program code is executable by the client device to cause the client device to transmit one or more response objects encoding a response and further responses to the server system;

the server system receiving the one or more response objects from the client device and determining the response and further responses encoded in the one or more response objects;

a clustering module of the server system identifying one or more clusters of sleep disorder user data that most closely relate to the determined response and determined further responses;

a recommendation module of the server system identifying a sleep disorder based on the determined response, determined further responses and the identified one or more clusters;

the recommendation module generating one or more recommendations based on the identified sleep disorder, the determined response, the determined further responses and the identified one or more clusters; and

determining a priority of each of the one or more recommendations based on: the identified one or more clusters of sleep disorder user data;

the server system encoding the generated one or more prioritized recommendations in a recommendations object and making the recommendations object accessible to the client device.

The query program code may comprise a query object and one or more further query objects; the response may be a response to the query object; the further responses may be responses to a subset of the one or more further query objects; and the subset of the one or more further query objects may be determined based on the response.

The determining of the subset of the one or more further query objects may be performed by the server system. Instead, the determining of the subset of the one or more further query objects may be performed by the client device.

At least one of the one or more further query objects may be directed to a sensor and at least one of the one or more response objects comprises health data from the sensor. The health data may comprise one or more of: cardiac activity measurement data, physical activity measurement data, blood pressure measurement data, respiratory activity measurement data, heart rate data, movement data, breathing sound data or breathing rate data.

The identified sleep disorder may be a primary sleep disorder and the recommendation module may further identify one or more secondary sleep disorders based on the determined response, the determined further responses and the identified one or more clusters.

The clustering module may perform clustering based on any one of: similarity learning, distance metrics, feature vector comparison or agglomerative clustering.

At least one of the one or more response objects may comprise free text and a natural language processing module of the server system is configured to process the free text to determine an input to the recommendation module.

The method may further comprise:

receiving feedback input via the client device regarding the one or more recommendations;

reconfiguring a recommendation model of the recommendation module to take into account the received feedback input.

Some embodiments relate to a computer-implemented method for revising sleep disorder related recommendations based on adherence, the method comprising:

a server system transmitting query program code to a client device, wherein the query program code is executable by the client device to cause the client device to transmit one or more response objects encoding a response and further responses to the server system;

the server system receiving the one or more response objects from the client device and determining the response and the further responses encoded in the one or more response objects, wherein at least one of the further responses comprises health data from a sensor associated with a user of the client device;

a recommendation module of the server system identifying a sleep disorder based on the determined response, and the determined further responses;

the recommendation module generating one or more recommendations based on the identified sleep disorder, the determined response, and the determined further responses; and

determining one or more adherence metrics based on a comparison between the generated one or more recommendations and the health data;

the recommendation module generating one or more revised recommendations based on the one or more adherence metrics; and

the server system encoding the generated one or more revised recommendations in a recommendations object and making the recommendations object accessible to the client device.

The query program code may comprise a query object and one or more further query objects; the response may be a response to the query object; the further responses may be responses to a subset of the one or more further query objects; and the subset of the one or more further query objects may be determined based on the response object.

The determining of the subset of one or more further query objects may be performed by the server system. Instead, the determining of the subset of one or more further query objects may be performed by the client device.

The health data may comprise one or more of: cardiac activity measurement data, physical activity measurement data, blood pressure measurement data, respiratory activity measurement data, heart rate data, movement data, breathing sound data or breathing rate data.

The recommendation module may further identify one or more secondary sleep disorders based on the determined response and the determined further responses.

The method may further comprise the step of:

a clustering module of the server system identifying one or more clusters of sleep disorder user data that most closely relate to the determined one or more adherence metrics; and

wherein the generation of one or more revised recommendations is also based on the identified one or more clusters of sleep disorder user data.

The clustering module may perform clustering based on any one of: similarity learning, distance metrics, feature vector comparison or agglomerative clustering.

At least one of the one or more response objects may comprise free text and a natural language processing module of the server system is configured to process the free text to determine an input to the recommendation module.

The method may further comprise:

receiving feedback input via the client device regarding the one or more recommendations;

reconfiguring a recommendation model of the recommendation module to take into account the received feedback input.

The one or more adherence metrics may comprise one or more of: total sleep time, bed time regularity, time in bed, or sleep hygiene measure.

Some embodiments relate to a computer-implemented method for determining sleep disorder recommendations and quality of life metrics:

a server system transmitting query program code to a client device, wherein the query program code is executable by the client device to cause the client device to transmit one or more response objects encoding a response and further responses to the server system;

the server system receiving one or more response objects from the client device and determining the response and further responses encoded in the one or more response objects wherein at least one response object comprises health data from a sensor associated with a user of the client device;

a recommendation module of the server system identifying a sleep disorder based on the determined response and determined further responses;

the recommendation module generating one or more recommendations based on the identified sleep disorder, the determined response and the determined further responses;

determining one or more quality of life metrics based on the determined response or further responses or the health data of the user; and

the server system encoding the generated one or more recommendations and one or more quality of life metrics in a recommendations object and making the recommendations object accessible to the client device.

The method may further comprise a clustering module of the server system identifying one or more clusters of sleep disorder user data that most closely relate to the determined one or more quality of life metrics; and the generation of one or more recommendations may be further based on the identified one or more clusters of sleep disorder user data.

The clustering module may be further configured to determine a rate of change of each quality of life metric for each of the determined one or more recommendations.

The query program code may comprise a query object and one or more further query objects; the response may be a response to the query object; the further responses may be responses to a subset of the one or more further query objects; and the subset of the one or more further query objects may be determined based on the response object.

The determining of the subset of one or more further query objects may be performed by the server system. Instead, the determining of the subset of one or more further query objects may be performed by the client device.

The health data may comprise one or more of: cardiac activity measurement data, physical activity measurement data, blood pressure measurement data, respiratory activity measurement data, heart rate data, movement data, breathing sound data or breathing rate data.

The recommendation module may further identify one or more secondary sleep disorders based on the determined response and determined further responses.

The clustering module may perform clustering based on any one of: similarity learning, distance metrics, feature vector comparison or agglomerative clustering.

At least one of the one or more response objects may comprise free text and a natural language processing module of the server system is configured to process the free text to determine an input to the recommendation module.

The method may further comprise:

receiving feedback input via the client device regarding the one or more recommendations;

reconfiguring a recommendation model of the recommendation module to take into account the received feedback input.

Some embodiments relate to a computer-implemented method for branching-based recommendation generation regarding sleep disorders, the method comprising:

a server system transmitting a query object to a client device;

the server system receiving a response object from the client device in response to the query object and determining a response encoded in the response object;

the server system transmitting further query objects to the client device based on the determined response;

the server system receiving one or more further response objects from the client device;

the server system determining further responses encoded in the one or more further response objects;

a first recommendation branch of a recommendation module of the server system determining a first set of recommendations based on the determined response and the determined further responses;

the server system receiving one or more data objects comprising health data from a sensor associated with a user of the client device;

a second recommendation branch of the recommendation module determining a second set of recommendations based on the one or more data objects comprising the health data; and the server system encoding the generated first and second set of recommendations in a recommendations object and making the recommendations object accessible to the client device.

The method may further comprise determining a priority of each of the recommendations in the first and second set of recommendations.

At least one of the query object and the one or more further query objects may be directed to a sensor associated with a user of the client device and at least one of the one or more response objects comprises health data from the sensor.

The health data may comprise one or more of: cardiac activity measurement data, physical activity measurement data, blood pressure measurement data, respiratory activity measurement data, heart rate data, movement data, breathing sound data or breathing rate data.

The recommendation module may further identify one or more primary sleep disorders and one or more secondary sleep disorders based on at least the determined response, and the determined further responses.

At least one of the one or more response objects may comprise free text and a natural language processing module of the server system is configured to process the free text to determine an input to the recommendation module.

The method may further comprise:

receiving feedback input via the client device regarding the one or more recommendations;

reconfiguring a recommendation model of the recommendation module to take into account the received feedback input.

Some embodiments relate to a computer-implemented method for cluster-based adjustment of recommendations regarding sleep disorders, the method comprising:

a server system transmitting query program code to a client device, wherein the query program code is executable by the client device to cause the client device to transmit one or more response objects encoding a response to the server system;

the server system receiving the one or more response objects from the client device and determining the response and further responses encoded in the one or more response objects;

a clustering module of the server system identifying one or more clusters of sleep disorder user data that most closely relate to the determined response;

a recommendation module of the server system identifying a sleep disorder based on the determined response and the identified one or more clusters;

the recommendation module generating one or more recommendations based on the identified sleep disorder, the determined response and the identified one or more clusters;

the server system transmitting further query program code to the client device, wherein the further query program code is executable by the client device to cause the client device to transmit one or more further response objects encoding a further response to the server system;

the clustering module of the server system revising the identified one or more clusters of sleep disorder user data based on the determined further response;

the recommendation module of the server system revising the identified sleep disorder based on the determined further response and the revised identified one or more clusters;

the recommendation module generating one or more revised recommendations based on the revised identified sleep disorder, the determined further response and the revised identified one or more clusters;

the server system encoding the generated one or more revised recommendations in a recommendations object and making the recommendations object accessible to the client device.

At least part of the further query program code may be directed to a sensor associated with a user of the client device and at least one of the one or more further response objects comprises health data from the sensor. The health data may comprise one or more of: cardiac activity measurement data, physical activity measurement data, blood pressure measurement data, respiratory activity measurement data, heart rate data, movement data, breathing sound data or breathing rate data.

The recommendation module may further identify one or more secondary sleep disorders based on the determined response, the identified one or more clusters and/or the revised identified one or more clusters.

The clustering module may perform clustering based on any one of: similarity learning, distance metrics, feature vector comparison or agglomerative clustering.

At least one of the one or more response objects may comprise free text and a natural language processing module of the server system is configured to process the free text to determine an input to the recommendation module.

The method may further comprise:

receiving feedback input via client device regarding the one or more recommendations;

reconfiguring a recommendation model of the recommendation module to take into account the received feedback input.

Some embodiments relate to a system for information processing regarding sleep disorders, the system comprising a server system comprising:

one or more processors;

a memory accessible to the one or more processors storing executable program instructions to implement the method of any one of the embodiments described above or herein.

Some embodiments relate to computer-readable media storing computer-executable instructions that, when executed, direct one or more computers to perform the method of any one of the embodiments described above or herein.

### BRIEF DESCRIPTION OF THE FIGURES

FIG. 1 is a block diagram of a decision support system regarding sleep disorders;

FIG. 2 is a flowchart of a method for determining recommendations regarding sleep disorders;

FIG. 3 is a flowchart of a method for determining recommendations regarding sleep disorders by taking into account sensor data;

FIG. 4 is flowchart of a method for determining recommendations regarding sleep disorders by relying on more than one recommendation branch;

FIG. 5 is flowchart of a method for determining recommendations regarding sleep disorders by taking into account quality of life metrics;

FIG. 6A to 6D are example screenshots of a client device application according to some embodiments; and

FIG. 7 is a schematic diagram of a part of the decision support system according to some embodiments.

### DETAILED DESCRIPTION

The embodiments generally relate to decision support systems and methods for sleep disorder identification. Some embodiments rely on information from sensor devices such as fitness or health related sensor devices to provide improved identification of sleep disorders or more relevant recommendations. Some embodiments, after providing an indication of sleep disorders and relevant recommendations to a specific user, keep track of the adherence or engagement of the user to the recommendations and assess the effectiveness of the recommendations provided earlier. Some embodiments identify clusters of users based on information available regarding the users and rely on the identified clusters to provide improved indication of sleep disorders and relevant recommendations to a specific user. Some embodiments rely on the feedback of the users regarding their recommendations and subsequent changes to the users' quality of life to provide improved recommendations.

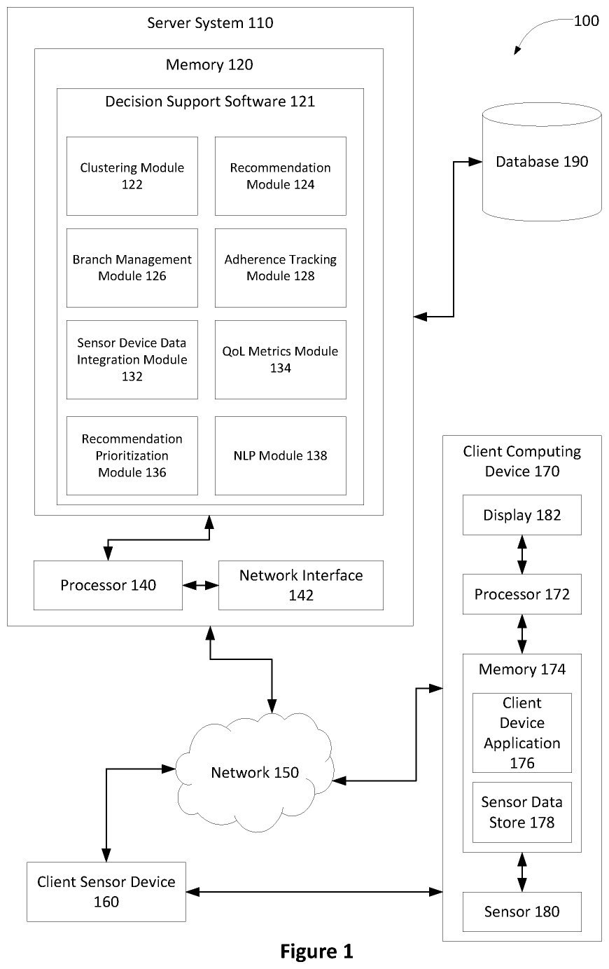

FIG. 1 is a block diagram of a decision support system 100 according to some embodiments. Decision support system 100 comprises a server system 110 that implements a server side part of decision support software 121. A client computing device 170 implements the client side part of the decision support software. More than one client computing device 170 may interact with the server system 110 to form a part of the decision support system 100. In some embodiments, a client sensor device 160 may also form part of the decision support system 100. In some embodiments, the client computing device 170 may comprise a sensor data store 178. The sensor data store 178 may store data generated by the client sensor device 160 and/or a sensor 180 that is part of the client computing device 170. The sensor 180 may be or comprise an audio sensor or a health data tracking sensor that gathers information regarding health data of the end user, for example. The client sensor device 160 may be or comprise a health or sleep related sensor or a fitness tracker, for example. The client sensor device 160 may be a smartwatch, for example, that tracks an individual's health data over time.

The health data may comprise one or more of: cardiac activity measurement data, physical activity measurement data, blood pressure measurement data or respiratory activity measurement data, or other longitudinal biological measures (i.e. gathered over a period of time of days, weeks or months), for example. Cardiac activity measurement data may include a heart rate measurement, a measure of electrical activity of the heart, or a measure of volumetric changes of the heart, for example. Physical activity measurement data may include measures of physical activity, for example such as: distance moved, steps taken, and/or calories burnt. Respiratory activity measurement data may include breathing rate per minute or lung volume change measurement, for example.

The client computing device 170 may be an end user computing device, such as a laptop or a tablet or a PC or a smart phone, for example. The client computing device 170 comprises a processor 172 and a memory 174. The client device application 176 is implemented within memory 174 that allows an end user to interact with the decision support software 121. The client device application 176 may include dedicated local application software (known as an “app”) or a browser application executing on the client computing device 170. The client computing device 170 also comprises a display 182 that enables the display of information to an end user and, in some embodiments, the entry of responses by the end user.

The system server 110 implements the client side part of the decision support system 100 through various software modules implemented in the memory 120. The server system 110 comprises a processor 140 and a network interface 142.

The various modules implemented in the server system 110 include, for example: a clustering module 122 that performs the function of clustering information; a recommendation module 124 that performs the function of generating recommendations based on information; a branch management module 126 that performs the function of managing information processing branches according to some embodiments; an engagement tracking module 128 that performs the function of tracking the engagement of end users with the decision and support software; a sensor device data integration module 132 that specifically handles information generated by sensors held by end-users or associated devices, such as client sensor device 160 or client computing device 170; a quality-of-life metrics module 134 that determines quality-of-life metrics based on available information; a recommendation prioritisation module 136 that performs the prioritisation of recommendations generated by the recommendations module 124; and an NLP module 138 that performs the function of natural language processing to derive information from natural language data. The recommendation module 124 may comprise one or more models or branches for generating recommendations or for identifying sleep problems based on input. Some of the models or branches may rely on the information in database 190 to generate the recommendations or to identify sleep problems.

The network interface 142 may comprise software or hardware or a combination of software and hardware enabling the communication of the server system 110 with multiple client computing devices 170 and multiple client sensor devices 160. A database 190 may also form part of the decision support system 100 and may perform the function of acting as a repository for information gathered by the server system 110 and also metrics and inferences generated by the server system 110. The database 190 may comprise a conventional relational database or a NOSQL database implemented on a separate server system. In some embodiments the database 190 may be implemented within server system 110. The server system 110 in some embodiments may be implemented on one or more virtual servers available as part of the cloud implementation. Likewise database 190 in some embodiments may be implemented as part of a cloud service implementation.

Database 190 comprises sleep disorder user data for a population of individuals. The sleep disorder user data may comprise data collected from sensors regarding the sleep patterns or other health related data capable of enabling inferences regarding sleep quality. The sleep disorder user data may also comprise information specific sleep disorders and efficacy of recommendations and treatments for individuals. The sleep disorder user data may provide a population of user data suitable for clustering, whereby sleep disorder user data of individuals may be allocated to one or more predetermined clusters depending on one or more sleep disorder, sleep pattern or sleep behaviour-based attributes. In some embodiments, user data relating to the demographics of the user may be used to allocate a user to one or more clusters based on demographics of all users. User data relating to demographics may include: age, sex, education level, income level, marital status, occupation, for example.

Each cluster may represent a set of individuals that share some common sleep disorder related attributes or data. The sleep disorder user data of database 190 may serve as a data set for clustering any new sleep disorder user data not previously stored in the database 190. As the sample of sleep disorder user data held in the database 190 grows over time, the predetermined clusters may be revised to take into account new sleep disorder user data received in the database 190. The revision of the predetermined clusters may comprise: identification of new clusters, merging of existing clusters, and/or reallocation of existing sleep disorder user data to a different cluster, for example. The clustering may be performed using clustering techniques including one or more of: similarity learning, distance metrics, feature vector comparison, k-mean, density-based, expectation-maximisation, mean-shift or agglomerative clustering techniques, for example.

A network 150 may include, for example, at least a portion of one or more networks having one or more nodes that transmit, receive, forward, generate, buffer, store, route, switch, process, or a combination thereof, etc. one or more messages, packets, signals, some combination thereof, or so forth. The network 150 may include, for example, one or more of: a wireless network, a wired network, an internet, an intranet, a public network, a packet-switched network, a circuit-switched network, an ad hoc network, an infrastructure network, a public-switched telephone network (PSTN), a cable network, a cellular network, a satellite network, a fiber optic network, some combination thereof, or so forth.

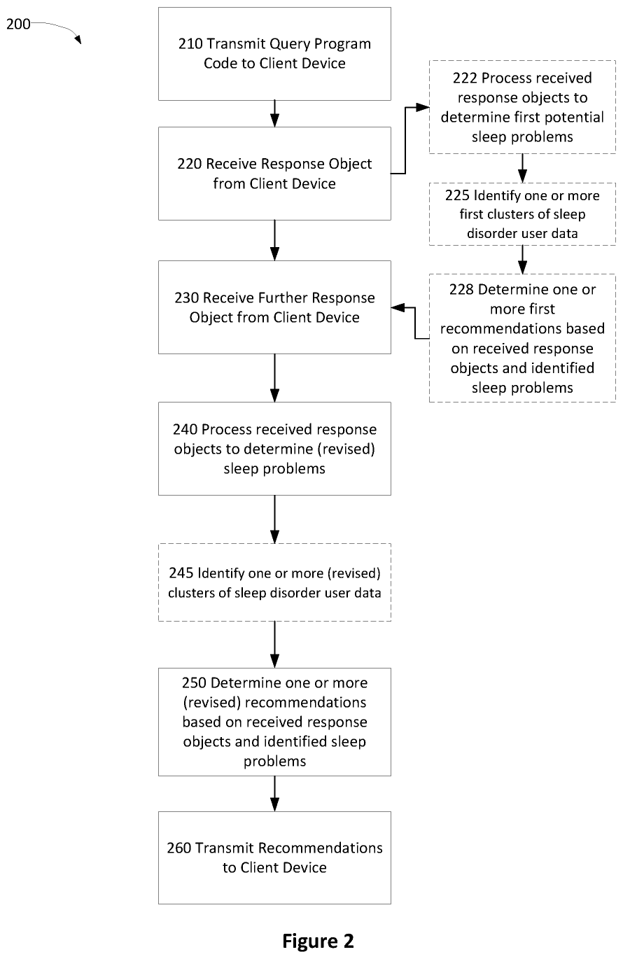

FIG. 2 illustrates a method 200 showing some of the steps implemented by the decision support software 121 according to some embodiments. At step 210 of method 200, the server system 110 transmits query program code to the client computing device 170. The query object 210 may comprise an encoded directive or query requesting the client computing device 170 for information or a response in relation to the directive or query. The client computing device 170, after receiving the query object with an encoded directive, may display the directive to the end user through the client device application 176. The end user may provide a response to the directive through the client device application 176. The response may be encoded into a response object by the client device application 176 before transmitting it to the server system 110 over the network 150.

At step 220, the server system 110 receives the encoded response object from the client computing device 170 over the network 150. In some embodiments, option steps 222, 225 and 228 may be performed. Step 222 comprises processing of the response received at step 220 by the recommendation module 124 to determine a first sleep disorder. At step 225, one or more first clusters of sleep disorder user data may be identified by the clustering module 122. At step 228, one or more first recommendations may be determined by the recommendations module 124 based on the determined first sleep disorder and the identified first cluster. The optional steps 222, 225 and 228 rely only on the received response object at step 220 to perform the necessary information processing.

At step 230, the decision support software 121 may receive further response objects from the client computing device 170. In some embodiments, the query program code transmitted at step 210 may comprise a query object and one or more further query objects. In some embodiments, the response received through the response object 220 may determine a subset of the further query objects to be presented through the client computing device to receive the further response objects (in response to the subset of further query objects). The subset of further query objects may comprise less than all possible further query objects that are stored in database 190. The determination of the subset of query objects to be presented may occur on the server system 110. Alternatively, the determination of the subset of query objects to be presented may occur on the client device 170. In effect, the determination of a subset of query objects may provide the advantage of making the method 200 more efficient by potentially shortening it.

The cadence of the iterative process implemented through steps 210 to 230 can be accelerated by shortening the flow (such that only the most relevant query objects are transmitted to the client device 170) and increasing the frequency of the entire method 200 (e.g. daily instead of weekly). By leveraging a large user population enrolled in use of the decision support system 100, the convergence to a list of optimally ordered recommendations can be accelerated. An example of a relevant query object may include a question for a patient undergoing treatment for primary sleep onset insomnia that includes a directive about subjective sleep latency or objective sleep latency, should the patient have a sleep tracking device.

In considering the most relevant query objects for a given sleep disorder or treatment, it is also possible to include some redundancy to ensure that the information provided by the response objects is maximally reliable. For instance, if sleep duration is an important metric to determine treatment effectiveness, one may elect to ask: 1) the subjective sleep duration and 2) bedtime and wakeup time. The latter can be used by the decision support software 121 as a control on the reliability of the answer to question 1 (e.g. a subjective sleep duration of 6 hours may not be consistent with subjective bedtime/wake-up time of midnight and 4 am respectively).

At step 240, the decision support software 121 processes the response objects received at step 220 to determine sleep problems based on the encoded information in the response objects. Determination of the primary and one or more secondary sleep problems is performed by the recommendation module 124. A numeric value or relevance of each identified sleep disorder may be calculated by the decision support software 121, allowing the relative ranking of each identified sleep disorder in terms of relevance. The recommendation module 124 may invoke other modules of the decision support software 121, depending on the various embodiments of the decision support software 121. In embodiments where step 222 is performed, the sleep disorder or problem identified at step 240 may be a revised sleep disorder or problem based on the further query objects transmitted at step 230.

In some embodiments, an optional step 245 of identifying one or more revised clusters of sleep disorder user data may be performed. This step may involve a revision of the one or more first clusters identified at step 225.

At step 250, the recommendation module 124 determines, based on the identified primary and secondary sleep problems, recommendations to address identified problems. The recommendation module 124 may perform the operation of identifying primary and secondary sleep problems and recommendations based on various computational methodologies, including decision trees (in particular random forest decision trees), support vector machines, artificial neural networks, models of sleep problem identification and recommendation generation or other artificial intelligence-based computational models, for example.

At step 260, the decision support software 121 transmits a recommendation object to the client computing device 170. The recommendation object comprises encoded recommendations generated by the recommendations module 124 applicable to the end user of the client computing device 170. The client device application 176 processes the encoded recommendations and displays to the end user the recommendations of the recommendations object received by the client computing device 170. The recommendation module 124 may use any and all available data or information about the current user, including but not limited to: subjective data, responses to the clinically validated surveys regarding sleep, responses to additional transmitted query objects, predicted sleep conditions for the user based on the rest of the information, and objective data including numeric metadata about when, how, and how long the user took to complete the response process or a specific part of the response process, time of the day the user responded, time zone and country of the user, for example.

In some embodiments, the transmitted query program code at step 210 may comprise query objects based on an insomnia severity index (ISI) determination questionnaire or alternatively Pittsburgh sleep quality assessment index (PSQI) questionnaire. In some embodiments, the transmitted query program code at step 210 may comprise query objects based on a shift work disorder questionnaire, for example. In some embodiments, the transmitted query program code at step 210 may comprise query objects based on an OSA-50 screening questionnaire, a Berlin Questionnaire, a STOP-BANG questionnaire, a Karolinska Sleepiness Scale (KSS) questionnaire, a Stamford Sleepiness Scale questionnaire, a Flinders Fatigue Scale questionnaire and/or a Fatigue Severity Scale questionnaire, for example.

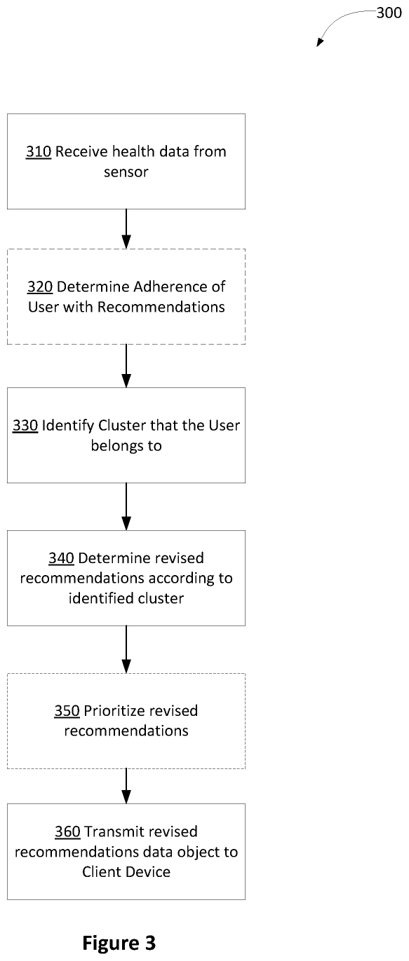

FIG. 3 is a flowchart of a method 300 implemented by the decision support system 100 to process sensor data objects received from client sensor device 160 or client computing device 170. At step 310, the server system 110 receives client sensor data objects over the network 150. The client sensor data objects comprise encoded client sensor data sensed by either the client sensor devices 160 or sensor 180 in relation to an end user over a particular (predetermined or predefined) period of time.

At step 320, the decision support software 121 optionally determines or estimates the adherence or compliance or engagement of the end user to a recommendation based on the received sensor data objects. The recommendations providing the benchmark for assessment of adherence or engagement may have been previously generated by the decision support software 121, for example by the method of flowchart 200. The determination of adherence may comprise comparing the encoded sensor data in the sensor data objects to expected sensor data based on previously generated recommendations, for example. In some embodiments, the adherence may be determined by comparison to a default recommendation standard. The determination of the adherence may be performed by the engagement tracking module 128.

The determination of deviation from adherence or engagement may be accessed by quantifying program or recommendation deviation including by determining the numerical difference between user behaviours, and the recommendations determined at step 250. Non-limiting examples of factors used in the determination of deviation include the following: total sleep time difference, bed time or wake-up time regularity, time in bed, and sleep hygiene.

The above factors for determination of deviation may be determined based on one or more sub-factors, as described below, by way of example. Total Sleep Time Difference comprises the difference between observed and recommended totals, derivative of observed total (since treatment began), and variance of observed totals over a historical window. Bed Time Regularity comprises the difference between observed and recommended bed times, and bed time variabilities (variances) over a historical window. Time In Bed comprises the difference between observed and recommended totals, derivative of observed total (since treatment began), and variance of observed totals over a historical window. Sleep Hygiene comprises the difference between observed and recommended device avoidance, and exercise and activity avoidance within some threshold of time before bed. The determination of deviation or adherence or engagement may also take into account data relating to the above parameters acquired over time.

At step 330, based on the received sensor data object, an identification of a cluster of users from among a predetermined set or group of clusters of users, to which the end user belongs may be performed. Once a user provides one or more responses to the query objects, data associated with that user is stored into database 190 and then, using the clustering module 122, the user is associated with one or more clusters. Step 330 is performed by the clustering module 122. At step 340, the decision support software 121 then determines a revised recommendation according to the identified cluster to which to end user belongs. The revised recommendation may be a recommendation that has exhibited a greatest statistical significance with respect to the cluster of users to which the specific end user has been identified to belong to. The revised recommendations in some embodiments may also be based on the adherence to previous recommendations of the end user determined at step 320. The step 340 may be performed by the recommendation module 124.

Each predetermined cluster of users may represent correlated groups of users which exhibit some form of similarity in relation to sleep related behaviour or metrics. One example of a predetermined cluster is a cluster or people who are found to have significantly high work-hours-per-week, and very short sleep. One recommendation for users relating to this predetermined cluster may relate to better work life balance.

In some embodiments, the step 340 may comprise a calculation of the correlation between the information received from response objects received at step 220 and the information received from the sensors 160 or 180. This may allow the assessment of the relation between or the difference between the perception regarding a particular sleep metric and sensor data obtained representing the sleep metric. The calculation of the correlation can occur for a single user, or for a subset of users, or the entire population of users. The calculated correlation may have a specific meaning depending on the sleep disorder under consideration. For example, insomnia most often is characterised by a mismatch between the actual sleep duration and perceived sleep duration by an individual. For users suffering from insomnia, the correlation between a response object conveying sleep duration and sensor data conveying sleep duration would be lower than expected, for example.

The recommendation module 124 may combine the information obtained through the received client sensor device data objects and any received query response objects to form an initial representation of the user (which can be in the form of a feature vector, for example). Similar information is used to represent all users of the decision support system 100 along with information about which solutions or recommendations (and effectiveness thereof) those users chose to use to address the identified sleep problems.

Unsupervised machine learning, similarity learning, distance metrics, and/or other feature vector comparison techniques may be used to determine a cluster or subset of users to which the current user is most similar. For example, a vector quantization algorithm, or Euclidian distance based comparison, or cosine similarity based comparison may be used to cluster users based on feature vectors.

Based on the similarities determined by the clustering module 122, the recommendation module 124 generates an initial set of sleep solution suggestions or recommendations. These recommendations may be transmitted to the client computing device 170 associated with the user. The user may then have the opportunity to give thumbs up/thumbs down style feedback on the recommendations, click links to view the solutions on other sites, read reviews and feedback from other users, and read the reasons that these recommendations have been presented to him/her, etc.

In some embodiments, the recommendation module 124 may receive updated information regarding a user, either in the form of a response to a follow-up query object or updated client sensor device data, which may form the basis of a revised or updated recommendation at step 360.

In relation to steps 260 and 360 of flowchart 200 and 300 respectively, the recommendation module 124 records interaction parameters of the user with data objects presented to the user via client computing device 170. For example, the recommendation module 124 may record any of: time spent viewing each recommendation, whether the user clicked on particular recommendations and whether the user indicated approval (thumbs up) or disapproval (thumbs down) for specific recommendations. The recommendation module 124 may also make determinations based on a user trajectory, for example including: revised responses to the initial or additional query objects and revised prediction of sleep conditions.

In some embodiments, the method 300 may be performed by a user to share results or logs from consumer or medical sensors frequently used by the patient such as the client sensor device 160 or sensor 180. Signals and response objects are analysed to derive compliance, adherence, and similarity quantifiers. This information is optionally integrated with response objects obtained at step 220 and/or signals from a cluster of users that are similar (according to a given metric) to the target user. The clustering process may leverage a population of existing and former users enrolled in the decision support system 100.

Compliance/Adherence or similarity quantifiers are integrated with the results of the recommendations determined at step 250 to readjust (if needed) the initial recommendations (e.g. identifying additional comorbidities) which can lead to recommendation modifications.

Some examples of adherence or engagement quantifiers include: time difference between recommended bedtime and actual bedtime, time difference between recommended sleep duration and actual sleep duration, and difference between recommended amount of physical activity and actual physical activity, and time of day at which the physical activity is performed. The adherence or engagement quantifiers may be determined by the received response determined at step 240. In some embodiments, the adherence or engagement quantifiers may be determined by the information encoded in the sensor device data object received at step 310.

Some embodiments may include an optional step 350 that comprises prioritisation of revised recommendations. Based on previously identified primary and secondary sleep problems, more than one revised recommendation may be generated for a specific end user. Different recommendations may have different levels of efficacy, for example depending on the particular characteristics of the end user or the cluster to which the user belongs or to the user's levels of adherence to recommendations. For example, the recommendation prioritisation module 136 may take into account the user's adherence level, the identified cluster to which the user belongs, and the user's primary and secondary sleep problems identified to prioritise the recommendations generated by the recommendation module 124. At step 360, the decision support software 121 may encode the generated recommendations into a recommendation data object. The encoded recommendation data object is communicated to the client device 170 over the network 150. The end user may access the encoded recommendations through the client device application 176, for example.

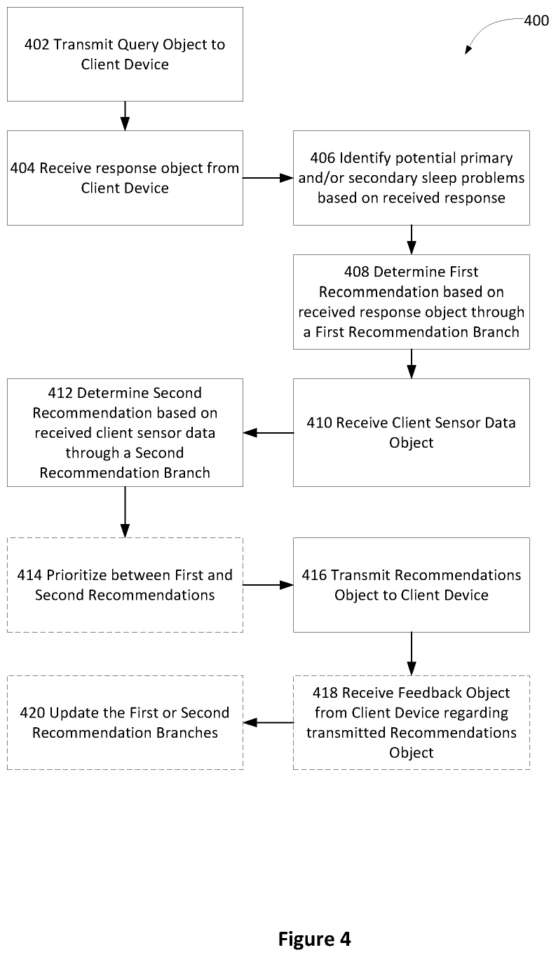

FIG. 4 is a flowchart of the method 400 of sleep disorder identification and recommendation generation implemented by the decision support software 121. At step 402, the query object is transmitted to the client device 170. The query object may comprise encoded directives requesting the client computing device 170 for information in response to the directives, for example. At step 404, a response object may be received from the client device 170 by the server system 110 in response to the query object transmitted at step 402. Based on the received response object, at step 406 the recommendation module 124 may identify potential primary and secondary sleep problems. At step 408, the recommendation module 124 may determine recommendations based on identified primary and secondary sleep problems. This determination of recommendations at step 408 may be performed by a first information processing branch implemented by the recommendation module 124.

At step 410, the server system 110 may receive client sensor device data objects from the client device 170 or the sensor 160 or a combination of both. At step 412, a second recommendation branch may be used to determine recommendations in response to the received client sensor data objects at step 410. The second recommendation branch is logically independent of the first information processing branch and thereby represents an independent method of processing information. The second recommendation branch also processes information received from the client sensor device 160 or the sensor 180 or both. This is in contrast to the first recommendation branch that relies on response objects received at step 404.

An optional step 414 may be performed by the prioritisation module 136 in order to prioritise the recommendations generated by the first recommendation branch and the second recommendation branch. At step 416, the decision support software 121 encodes the recommendations in a recommendations object and transmits the recommendations object to the client computing device 170.

In some embodiments, an optional step 418 may be performed to receive a feedback object from the client device 170. The feedback object may comprise an end user's feedback regarding the specific recommendations provided to the end user and may include an indication of the relevance or suitability of the recommendations generated. For example, the feedback may be positive or negative. At the further optional step 420, in some embodiments based on the received feedback, the first or second recommendation branches may be updated to reflect the suitability or relevance of the recommendations generated.

The method 400 performs sleep problem identification or classification using at least two (optionally 3, 4 or more) recommendation branches, each running separate classification algorithms, and subsequently leverages a combination of such branches to perform, in some embodiments, real-time automatic retraining of relevant machine learning components without offline intervention. All branches receive identical response object data, however only one branch may make further use of additionally available sensor generated data.

At least one response object based branch exists that provides a classification or indication of the user's sleep problem that is configured to closely follow the assessment of a typical physician.

At least one “data integration” based recommendation branch, such as the recommendation branch of step 412 exists that uses machine learning techniques in order to identify sleep problems and/or the ability to predict sleep problem development based on connections between different response objects and sensor data. The advantage of this multi-branch architecture lies in its ability to provide sleep disorder classifications or recommendations that may be similar to clinically validated classifications. Such classifications or recommendations can retain their clinical relevance over time as higher resolution and longitudinal data becomes available, and as the feature set used by the classification system is optimized.

In some embodiments, the determination at step 412 may take into account the response objects received at step 404. The first and second recommendation braches may leverage large populations of existing and former user data in the database 190 to learn patterns in data that correspond to each individual sleep problem and each combination of sleep problems. The second recommendations branch may produce a different set of classifications for a primary and secondary sleep problem than the first recommendation branch.

If no sleep problems are identified by either of the recommendation branches for a given user, the data collected may be used to evaluate if a user is at risk to develop any of the sleep problems identified by the system. A risk level threshold is determined by the recommendation prioritization module 136 and if above a certain threshold, those results may be shown back to the user.

The classifications from steps 408 and 412 are processed by the recommendation prioritization module 136 at step 414 to determine which sleep problem classifications are most likely to be more relevant and decide in which order to show the final set of a primary problem and secondary sleep problems to the user along with the relevant recommendations. The user then has the option to offer feedback about the recommendations at step 418. If the user gives feedback, the feedback can be used in step 420 to update the machine learning models implemented by the recommendation branches so that the recommendation prioritization module 124 and/or the recommendation prioritization module 136 can classify one or more sleep disorders of a patient more accurately. For example, if a particular recommendation receives approval from a significant number of users belonging to a particular cluster, that particular recommendation may be given greater priority over the rest of the recommendations for all current and future users belonging to the cluster. In some embodiments, there may be modifiable weights associated with the first and second recommendation branches.

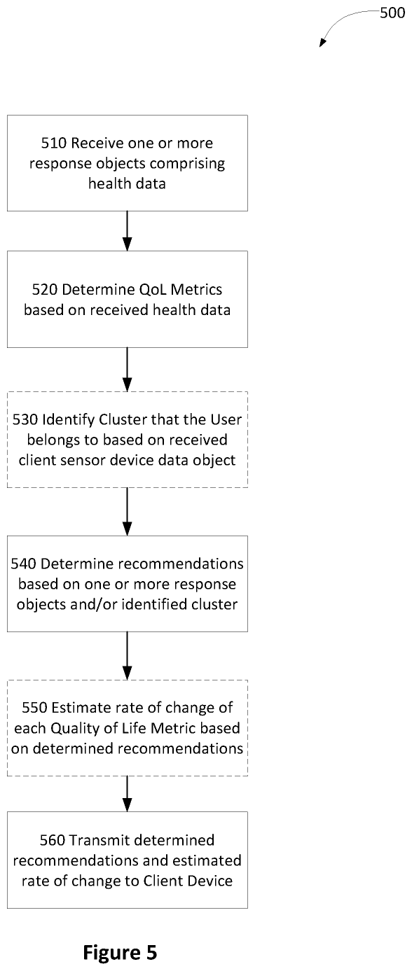

FIG. 5 is a flowchart of a method of sleep disorder identification and recommendation 500 that relies in part on determination of quality of life metrics. At step 510, the server system 110 receives a client sensor device data object. Based on the received client sensor device data object, at step 520, the quality of life metrics module 134 determines the quality of life metrics for the end user from which the client sensor device data object was received at step 510.

The client sensor device data object may be received periodically to gauge the impact of a previously presented recommendation on the quality of life of the user. A cluster of consumers or users that have similar sensor data, response objects from the user obtained at step 220 and models on how sleep impacts QoL are inputs to the QoL Metrics Module 128 to generate metrics on how sleep impacts the user's QoL.

For each monitored QoL metric, the QoL Metrics Module 128 keeps a log of the change history to track changes and rate-of-change during treatment. The information regarding rate of change may play an important role in assessing the progress of a user.

In some embodiments, the QoL Metrics Module 128 may determine both user and cluster metrics on how sleep impacts QoL. Cluster metrics may comprise overall metrics calculated for an identified cluster of users, for example. The cluster metrics may help users compare their QoL and sleep quality with the rest of the users in similar conditions. For example: the QoL Metrics Module 128 may provide concrete metrics via display 182 of client computing device 170 like ‘m’ number of people with condition ‘n’ followed treatment ‘o’ to solve their sleep issue and saw ‘p %’ improvement in their weight loss.

Personal metrics toward QoL goals are just as valuable to provide to help the user stay compliant to a recommendation. As the user improves and notices changes in their QoL metrics, it's easier for them to be compliant, knowing how sleep plays a broader role in their life and provides relatable measurements toward goals beyond improving sleep metrics.

The processes depicted in FIG. 5 can be iteratively repeated to quantify the impact of sleep disorder treatments on the quality of life to improve compliance and motivation to solve user's sleep problems.

At step 530, the clustering module 122 identifies a cluster of users to which the specific end user belongs, for example based on the received client sensor device data object. In some embodiments, the clustering module 122 may identify a cluster of users also based on the determined quality of life metrics at step 520.

At step 540, the recommendation module 124 determines recommendations for the end user based on the determined quality of life metrics and the identified cluster to which the end user belongs. In some embodiments, an optional step 550 may also be performed, involving an estimation of rate of change of each quality-of-life metric based on implementation of each recommendation. At step 560, the server system 110 transmits to the client computing device 170 the recommendations determined at step 540. Optionally, step 560 may also comprise transmission to the client computing device 170 of the estimated rate of change of each quality-of-life metric estimated at step 550.

In some embodiments, a part or whole of any step of each of the methods 200, 300, 400 or 500 could be performed on the client device. For example, in some embodiments, the step 230 could be performed on the client device, wherein as part of step 210, the client device receives further program code relating to further query objects and the client device may collate the response object and further response object before transmitting them to the server system 110.

Objects referred to in this specification could include data packets or executable code or interface calls capable of transmission over a communications network. Objects may comprise encoded information or executable code groups or segments that could be executed by a computing device to interact with the contents of an object.

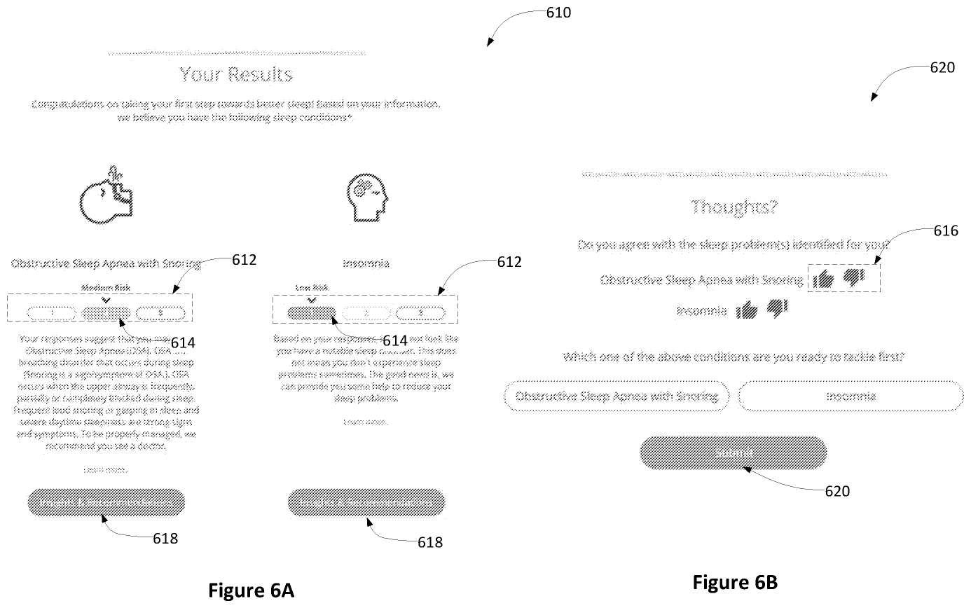

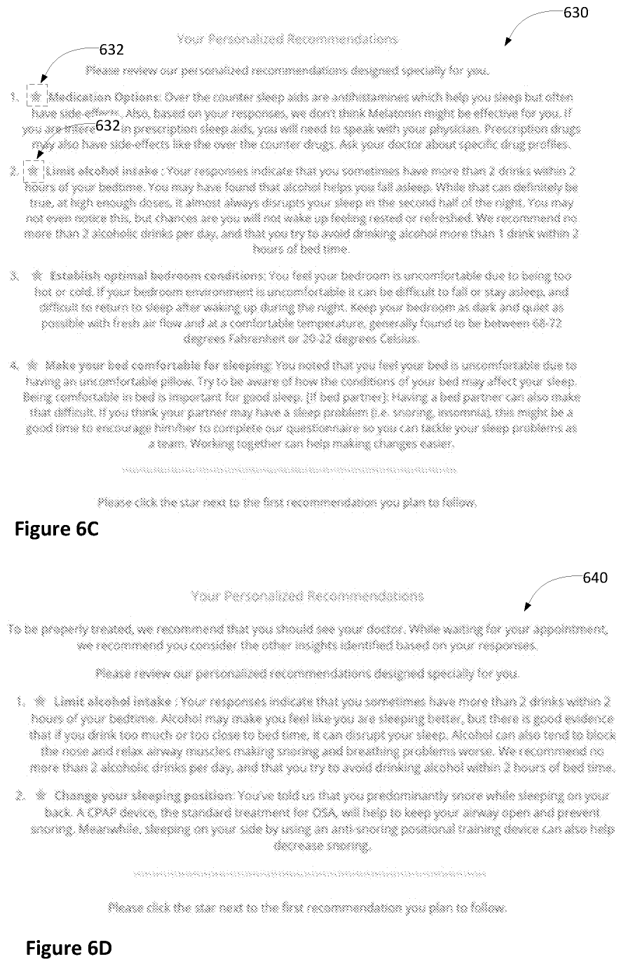

FIGS. 6A, 6B, 6C and 6D are example screenshots represented on the display 182 of the client computing device 170. FIG. 6A is an example screenshot display 610 showing a representation of the identified sleep problems in relation to an end user. Region 612 of display 610 represents the graded scale of risk level the respect to a particular sleep disorder. A risk level indicator 614 in region 612 indicates the level of risk of an end user with respect to an identified sleep disorder. The different displayed levels of risk may be low, medium or high, for example. The display 610 may also comprise a selectable icon 618 allowing the display of further insights and recommendations in relation to a particular sleep disorder identified in display 610.

Screenshot 620 of FIG. 6B represents an example interface display 620 at client computing device 170 allowing an end user to provide feedback input in relation to one or more identified sleep disorders on display 620. In some embodiments, the feedback input may be provided by clicking on thumbs up or thumbs down icons 616 in relation to each identified sleep disorder. Once the user has specified their feedback input at client computing device 170, for example using the selectable thumbs up or thumbs down icons 616, the user may click on the submit button 620 to submit the feedback input.

Screenshots 630, 640 in FIGS. 6C and 6D respectively illustrate an interface displaying recommendations in relation to identified sleep disorders for an end user. The interface as displayed in the screenshots 630 and 640 also includes selectable marker icons 632. The selectable marker icons 632 may allow an end user to subscribe to particular recommendation or provide feedback to the decision support system 100 in relation to a particular recommendation. Selection of such marker icons via the client computing device 170 is recorded by decision support software 121.

Some embodiments described herein relate to an objective, systematic and easily accessible method of identifying certain sleep disorders. Co-owned Australian provisional patent application no. 2018904007 describes related methods and the content of that application is incorporated herein in full by reference. Further, the described methods and systems are not affected by the background knowledge, preconceptions and emotional state of a human medical practitioner that may rely of the methods and systems of the embodiments. That is, described methods allows an assessment regarding sleep disorders to be conducted in a systematic, objective manner with minimal or no opportunity for relevant screening questions or a potential diagnosis to be overlooked though human error or subjectivity.

Part of the methods according to the embodiments may be performed as a computer application and other parts of the methods may be server-implemented, thereby allowing the method to be performed on various devices including desktop or laptop computers. Alternatively, part of the methods can be performed as a smartphone- or tablet-based application, which would allow for further flexibility by enabling the method to be performed almost anywhere and at any time. Part of the methods can also be performed as a web-based application through a hosted service provider, thereby allowing the method to be performed on any suitable device accessing the Internet. The method shall now be exemplified in respect of a preferred web-based application.

In some embodiments a user must log into a web-based decision support software 121 by providing a username and password. Upon an initial login the user may be asked for details such as age, gender and location for data collection and analysis purposes. After logging in to the decision support software 121, the user may be directed to a series of welcome pages before being directed to the recommendations module 124. By providing a login page, the user may revisit the decision support software 121 without having to re-input certain details, as well as having access to prior indications and sleep diary inputs of the user, as further discussed below.

It is intended that the decision support system 100 allow the user to receive an indication regarding sleep disorders as quickly and as simply as possible. In today's fast-paced society people are highly used to receiving information without having to provide unnecessary information or answer repetitive questions. Methods which are unduly lengthy or detailed therefore risk that users will abandon the method part-way though. The decision support system 100 is thereby designed to dynamically select directives or questions for presentation to the user based on responses to previous directives or questions, thereby ultimately reducing the number of directives or questions that the user must respond to.

To most simply exemplify this approach, the decision support system 100 may arrange the directives or questions to initially present triage-style questions enabling the decision support system 100 to immediately discount particular sleep disorders. As a very simple example, the decision support software 121 may generate a display to ask whether the user performs shift work. If the user replies ‘YES’, the decision support software 121 will generate a display to present further questions exploring the nature of the shift work. If the user replies ‘NO’, the decision support software 121 may discount shift work as an issue and move to another line of questioning.

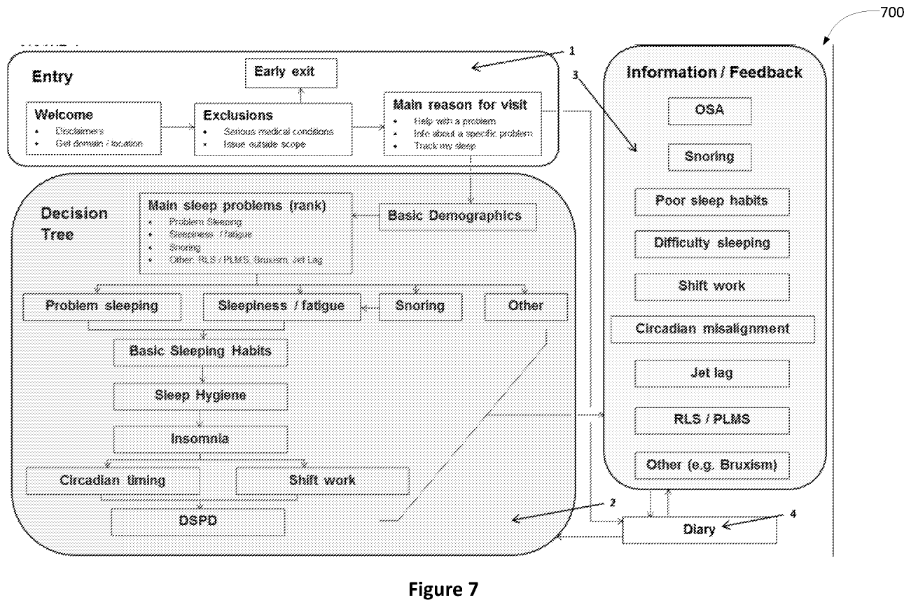

FIG. 7 illustrates a diagram of some parts 700 of the decision support system 100 according to some embodiments. In FIG. 7, the decision support system 100 is thematically divided onto three areas, being: the ‘Entry’ 1 or welcome pages, the ‘Decision Tree’ 2 pages in which the query objects are presented, and the ‘Information/Feedback Pages’ 3. These areas are now discussed in turn.

Within the entry or welcome pages, the user may be welcomed to an interface of the decision support system 100 and receive an explanation of options available to the user, including the option of receiving an indication of particular sleep disorders determined by system 100 to be relevant for the user. The user may also be presented with standard disclaimers explaining matters such as that indications in accordance with the decision support software 121 may not be suitable for those suffering serious medical conditions including lung or heart disease, asthma, or severe depression or anxiety. User information may also be extracted by, for example, accessing cookies present on the client computing device 170. Such information may include the location of the user and web browsing and search history of the user. This information may be used by the decision support system 100 for broader statistical analysis and, if applicable, could be used to assist in formulating an indication regarding sleep disorders. As an example, if a user's search history shows that they have recently researched snoring, this information may assist the decision support system 100 in determining which questions should be presented to the user. Alternatively, the location of the user may be used in broader epidemiological research.

The next stage of the Entry area allows the application to in effect triage users who suffer from serious medical conditions. The page may therefore ask whether the user is currently:

a) under the care of a physician for depression and/or anxiety,
b) taking prescription medications which may cause drowsiness, or
c) experiencing unpleasant leg sensations that cause an urge to move his or her legs, predominantly in the night.

If the user replies to any of these question with ‘YES’, the decision support system 100 may direct the user to seek medical attention and exit the application, or otherwise present a further disclaimer urging the user to seek medical attention and allow the user to continue through the application on that basis.

Once the user has navigated through the ‘Exclusions’ webpage, the user may be directed to next page which asks the user for the main reason for their visit to the website. In this example, a user may not actually require or be seeking an indication regarding sleep disorders, but rather merely be seeking general information on certain sleep disorders. If so, the application may allow the user to bypass the questionnaire, or ‘Decision Tree’ area of the decision support system 100, and direct the user to the information pages on particular sleep disorders. Similarly, the user may simply wish to make use of the sleep diary functionality of the website 4 (see FIG. 7), in which case the application will bypass the sleep disorder indication and the sleep disorder information pages, and direct the user to the sleep diary functionality.

In an alternative to what is shown in FIG. 7, the ‘main reason for visit’ page may present as part of the welcome to the website, thereby allowing the user obtain general information on sleep disorders without having to pass through disclaimer or exclusion protocols.

If the user seeks to obtain an indication regarding sleep disorders, the user will be directed to a demographics page that presents questions to receive basic demographic details of the user. Information sought may include the age, sex and occupation of the user, as well as how many hours a week the user works, and whether the user has bed partner or roommate. In an alternative to what is shown in FIG. 7, the information relating to basic demographics may be sought and obtained as part of the ‘Entry’ area, for example as part of the ‘Exclusions’ page, thereby reducing the number of pages visited by the user.

Other demographic information sought may include whether the user engages in shift work. If the user answers ‘YES’ to this question, the application may present further directives or questions allowing the person to detail and how many shifts the person has worked in the last month, as well as what types of shifts the person has worked (e.g. morning, afternoon, night, or extended duration).

The directives or questions may be presented in multiple choice formats. For example, one directive or question asks how many shifts the user has worked in the past month. If the user replies with ‘YES’ to this question, further questions seeking details on the extent of the shift work may be presented.

Once the information on the basic demographics of the user has been obtained, the decision support system 100 may direct the user to the next page in which potential sleep problems are identified and ranked. The questions asked may for example relate to whether the user:

a) has trouble falling asleep, staying asleep, or waking up too early;
b) experiences sleepiness that affects daily work or family life;
c) snores so as to disturb the sleep of the user or others;
d) is disturbed by the snoring of a partner or roommate.

In answering these questions the user may be asked to grade their issues in a scale from, for example, 1 to 10. This allows the user to quantify the effect or extent of a particular issue. The user's response will determine whether further questions are presented in relation to a particular issue, and will be used to form a diagnosis for the user. Having the user quantify his or her issues in this way also assist the application to assess the severity of a particular issue. For example, this approach may assist the application to distinguish between cases of mild snoring, heavy snoring, or obstructive sleep apnoea.

Further directives or questions presented at this stage may include:

a) how much sleep the user gets in a 24 hour period;
b) what time the user typically goes to bed;
c) how long during a main sleep period a user spends awake before finally waking up (i.e. getting out of bed); and
d) how many times a user usually wakes up during a main sleep period.

To determine the effect of work, or shift work, on sleep patterns similar questions may be asked in relation to weekend periods as follows:

a) how much sleep the user obtains on weekends;
b) what time the user usually goes to bed on weekends;
c) on weekends, how long during a main sleep period the user spends unable to sleep before finally waking up; and
d) on weekends, how many times the user usually wakes up during a main sleep period.

Continuing on the issue of shift work, the decision support software 121 may present further questions allowing the person to detail and how many shifts the person has worked in the last month, as well as what types of shifts the person has worked (e.g. morning, afternoon, night, or extended duration).

Having considered the above, the decision support system 100 may then present directives or questions relating to sleep habits, behaviours and environment. In particular, the application may initially ask how frequently the user engages in:

a) exercise before going to bed;
b) work or study, or sending or receiving emails, before going to sleep;
c) ‘bright light’ activities (such as using computers or smartphones) before going to sleep.
These directives or questions may again present as multiple choice, and allow the user to select between, for example, ‘often, ‘sometimes’, ‘rarely’ and ‘never’. Such questions may be presented because it is for example known that bright light before sleep may delay the release of melatonin, a sleepiness inducing hormone. Similarly, certain activities that may induce stress, anxiety or excitement can cause difficulty sleeping.

More specific directives or questions relating to the sleep environment may include whether the user engages in any of the following activities: watching television, reading, eating or drinking, studying, checking social media, or worrying.

The decision support system 100 may also present directives or questions to determine whether the user goes to bed or gets out of bed at different times of the day. The decision support system 100 may thereby for example determine whether the user habitually sleeps in past a regular wake up time. The decision support system 100 may also present directives or questions to determine whether the user regularly takes daytime naps, or more specifically whether the user regularly takes daytime naps of longer than two hours. The responses to such questions may help to identify disruptions to a stable circadian rhythm or to the homeostatic sleep drive.

The decision support system 100 may also present directives or questions about bed and bedroom conditions, For example, the application may ask whether the user's bedroom is uncomfortable due to being: too bright, too stuffy, too hot or cold, or too noisy. Similarly, the decision support system 100 may ask whether the user's bed is uncomfortable due to: too many blankets, uncomfortable pillow, or uncomfortable mattress. In addition, the application may ask whether the user has pets in the bedroom at night.

The decision support system 100 may also take account of the smoking and drinking habits of the user. For example, the decision support system 100 may ask whether, and how often, the user would drink caffeinated or alcoholic beverages, or smoke tobacco. The application may further ask when, for example, the user would have his or her last caffeinated beverage before going to bed. Alcohol consumption negatively affects sleep quality and can contribute to sleep disorders such as insomnia and obstructive sleep apnoea. As is well known, caffeine temporarily increases alertness and can result in difficulty falling asleep. Similarly, nicotine is a stimulant that can result in difficulty falling asleep.

The decision support system 100 may also assess the impact of any sleep difficulties on the user. Presented directives or questions may include whether the user's sleep difficulties are impacting the user's quality of life, or whether the user's sleep difficulties are interfering with daily activities or functioning. The decision support system 100 may also present directives or questions to determine how satisfied the user is with his or her sleep patterns.

The decision support system 100 may present further directives or questions to determine how fatigue is brought on or affects the user. The user may be asked to rate whether they agree that, for example:

a) motivation is affected when the user is fatigued;
b) exercise brings on his or her fatigue;
c) fatigue interferes with his or her physical functioning; or
d) fatigue interferes with his or her family or social life.

Following the above, the decision support system 100 may present directives or questions to determine how likely the user is to fall asleep during regular daytime activities such as: setting and reading, watching television, riding as a passenger in a car, or sitting quietly after lunch. These questions may ask the user to rate the likelihood from a scale of not likely to very likely.

The decision support system 100 may also ask directives or questions in part directed to whether the user suffers from delayed sleep wake phase disorder. The decision support system 100 may therefore ask whether the user:

a) has difficulty falling asleep as a desired bed time;
b) falls asleep easily if he or she stays up later; and
c) can sleep in easily.

The decision support system 100 may also ask questions directed to whether the user suffers from snoring/sleep apnoea. To gain an understanding of whether any weight issues apply, since obesity is for example a leading risk factor for obstructive sleep apnoea, the decision support system 100 may ask the user to provide his or her height and weight. The decision support system 100 may allow the user to provide a response to these questions in free-form (by typing in height and weight values). The application may also ask question around breathing and snoring, such as whether:

a) the user's snoring has ever bothered anyone;
b) anyone has seen the user stop breathing during sleep;
c) the user is currently being treated for high blood pressure;
d) snoring is mainly a problem for the user when he or she sleeps on his or her back; and
e) the user's nose is usually or often blocked.

These directives or questions may be presented as multiple choice, allowing the user to select between ‘yes’, ‘uncertain’ and ‘no’.

a) Other questions centred around snoring may include:
b) how often the user snores (e.g. every night, most nights, some nights, or rarely);
c) how much of the night the user snores (e.g. throughout the night, most of the night, some of the night, or rarely); and
d) how loud the snoring is (e.g. can be heard throughout the house, can be heard in the bedroom, and can barely be heard).

As discussed previously, to reduce the number of directives or questions presented to the user, the decision support system 100 will determine whether to present a question based on the user's response to an earlier question. For example, if the user indicates at an early stage that he or she does not snore, later questions directed to the extent or effect of any snoring may be avoided. Similarly, if the user initially indicates that he or she does not drink caffeinated beverages or smoke tobacco products, then questions directed to the extent of these activities can be avoided.

As noted previously, the application may present directives or questions in a manner enabling for free-form responses (e.g. as fully formed sentences). Natural language processing techniques implemented by the NLP Module 138 may then be used to identify keywords, sentiments, or other meanings to inform an initial assessment regarding sleep disorders and/or the selection of questions to present to the user. The NLP Module 138 may be trained on pre-annotated corpus data related to the topics of sleep and sleep problems. For example, natural language processing may be used to identify references or terms relating to snoring, and therefore present further and more detailed directives or questions relating to snoring as part of the questionnaire. The NLP module 138 may receive as input free form text or free text entered through the client device 170. The NLP module may also provide as output keywords, sentiments, or other signals to the recommendation module 124.

The decision support system 100 may also present directives or questions visually, in the sense that users must interact with an image in order to respond to the question. The user may be presented with a directive asking what time he or she goes to bed, and what time he or she gets out of bed. To respond to the questions, the user may click on a clock displayed by the display 182 as part of a query object transmitted by the server system 110. This approach has the advantage of keeping the directives or questions varied to avoid the user losing interest. Also, it can prevent the user from making typographical errors, since the response selected by the user can be clearly displayed to allow the user to identify when he or she makes an error. In addition, the decision support system 100 may not allow the user to reply with an illogical response, such as by not allowing the user to respond that he or she gets out of bed before actually going to bed. In addition, graphical representation allows for simple conveyance of other information, such as how long a user spends in bed following answering the exemplified questions.

Upon receiving responses to questions such as those above, the decision support system 100 should have sufficient information to make an indication about whether the user suffers from particular sleep disorders, such as insomnia (including sleep onset insomnia and sleep maintenance insomnia), snoring/obstructive sleep apnoea, delayed sleep wake phase disorder, chronic sleep restriction, and shift work disorder. Additionally, as shown in FIG. 7 and depending on the questions presented, the decision support system 100 may be suitable to provide an indication regarding poor sleep habits, jet lag, restless leg syndrome, periodic limb movement disorder, or other issues, such as sleep bruxism.

In presenting an indication of a sleep disorder, the decision support software 121 may weight and factor certain responses of the user to calculate a score in respect of a particular disorder. For example, to diagnose snoring/obstructive sleep apnoea, the application may add a predetermined number X of factor points (e.g. 3) where it determines that the user is over-weight. Similarly, the decision support system 100 may add a further X factor points where it determines that the user is occasionally woken up by his or her snoring, or a further X+Y (e.g. 3+2=5) factor points where it determines that the user is regularly woken up by his or her snoring. This accumulated score may then be used to, for example, determine whether the user suffers from mild, moderate, or severe snoring/obstructive sleep apnoea depending on whether the accumulated score reaches certain thresholds.

Depending on user responses, the decision support system 100 may indicate that the user may have more than one sleep disorder. In such cases the decision support system 100 may prioritise the indication of one disorder over another such that one disorder is identified as a primary disorder and one or more secondary disorders are also identified. The ranking of the sleep disorders may depend on the comparative points scored for each disorder as discussed above and/or it may depend on the relative health effects of the disorders. For example, snoring/obstructive sleep apnoea may be given priority over a shift work disorder, since it provides a higher risk of related health issues.

A decision support system 100 according to some embodiments was studied for validation by the present inventors. In particular, the decision support system 100 was used by 250 participants so as to compare diagnoses and/or recommendations presented by the application against those provided by a sleep specialist. Of those 250 participants, the primary sleep disorder indication provided by the decision support system 100 agreed with the primary diagnosis of the sleep specialist 81% of the time. This was considered as accurate as diagnoses provided by a typical medical practitioner. The decision support system 100 may therefore be used as a diagnostic tool to assist in sleep disorder diagnosis.

Depending on the sleep disorder indication presented to the user, the decision support system 100 may further present the user with information relating to the relevant disorder, as well as options for treatment. For example, if the user suffers from obstructive sleep apnoea, the decision support system 100 may explain that obstructive sleep apnoea is an obstruction of the airway during sleep resulting from relaxation of the tongue and airway muscles, and recommend that the user explore treatment options such as: weight loss, reduced alcohol consumption, cessation of smoking, using a mouth guard posture modification device or other oral appliance, or a ‘Continuous Positive Airway Pressure’ (‘CPAP’) device.

Alternatively, the decision support system 100 may recommend that a user arrange an appointment with a medical practitioner following presentation of a diagnosis. To that end, the decision support system 100 may provide the user with a list of nearby medical practitioners having necessary expertise in sleep disorders. Should the user elect to process in arranging an appointment, the analysis of the decision support system 100 may be forwarded to the relevant medical practitioner to aid the medical practitioner in assisting the user.

Further to the above, it is envisaged that the decision support system 100 could be used by a ‘first contact’ or primary care medical practitioner, to support a diagnosis made by that medical practitioner and avoid the medical practitioner feeling that he or she needs to refer the issue to a further sleep specialist.

Following completion of the analysis by the decision support system 100, the user may be asked if he or she would like to maintain a sleep diary. The sleep diary would allow the user to log information such as:

a) what time the user goes to sleep, and wakes up, each day;
b) whether, how often, and for how long, the user wakes up during his or her main sleeping period;
c) whether the user experienced any snoring, and the extent of any such snoring;
d) whether the user experienced fatigue affecting daytime activities;
e) whether the user fell asleep during the day, and for how long.

The sleep diary may also include information obtained from a client sensor device 160, such as a ‘FitBit’ ™, or an ‘Apple iWatch’ ™. Data obtained from the sleep monitoring device can be received and recorded as part of the sleep diary. For example, if the wearable client sensor device 160 is a FitBit™ wearable device, the application may automatically obtain sleep data by acquiring (and communicating via) API protocols for the device (and its related application(s)). This would avoid the user having to complete at least part of the sleep diary manually.

Data obtained from the sleep diary, or directly from the client sensor device and its related applications, may be used to confirm or adjust an indication provided pursuant to the responses to the directives or questionnaire. For example, if the user experiences snoring and falling asleep during the day, it may contribute to validating an indication of obstructive sleep apnoea. This information may in turn be used to improve of the accuracy of other assessments by the decision support software 121. In addition, if the data is provided before completing the directives or questionnaire, it may be used to reduce the number of directives or questions that need to be presented as part of the questionnaire. That is, if the user maintains a sleep diary, or uses a sleep monitoring device, prior to completing the questionnaire, information may be extracted to reduce the number of questions which need to be presented to the user during the questionnaire. For example, the user may not need to answer questions about how much sleep he or she averages per night, what time he or she goes to bed and wakes up, and how often and for how long the user wakes up during the night.

It is additionally anticipated that extraneous factors assessable while the user undertakes the questionnaire may be taken into account in assessing and presenting an indication regarding sleep disorders. For example, the decision support system 100 may record the time it takes to answer particular directives or questions. A delay in responding to a question, or questions on a certain topic, may inform whether the user is unsure of a certain answer. This may in turn inform the weight a particular answer is given in generating a diagnosis.

In addition, data obtained from the application may be used in further epidemiological studies. As a simple example, data obtained from the application may demonstrate that people of particular age, gender, state or city, or profession are more likely to suffer from a particular sleep disorder. This information may then be used to for example explore methods of preventative treatment, or to assist in the diagnosis of further sufferers.

To improve the accuracy of future indications regarding sleep disorders, users of the decision support system 100 may be invited to provide feedback in the form of comments or ratings. Where negative feedback is received, suggesting that the decision support system 100 may have provided an unsuitable indication regarding a sleep disorder, this may be used to update the recommendation models of the recommendations module 124.

Some embodiments incorporate methods of improving the indication regarding sleep disorders by utilising machine learning. In particular, machine learning allows evaluation and improvement of the indication regarding sleep disorders over time.

Machine learning comprises algorithms and techniques to autonomously train a model or branch to make indications or assessment. The method of improving the indications or assessments may involve supervised learning, in which relevant algorithms implements by the recommendation module 124, or recommendation prioritization module 136 or the NLP Module 138, for example are trained with examples of inputs (e.g. sleep disorder related data or health data) and corresponding outputs (e.g. sleep disorder indications or recommendations). With sufficient data and training the model can then, for example, be used for improved the quality or accuracy of the indications regarding sleep disorders. Machine learning of this type or similar can be used to determine which directive or question is the best choice to present next, given the responses to previously presented directives or questions. In addition, decision support system 100 may determine where the user may have answered a previous question inaccurately based on correlations obtained from previous users of the application. If this occurs, the decision support system 100 may present a similar question again to confirm the user's understanding, or give the response provided by the user lower weight.

Suitable machine learning techniques may involve, for example:

a) artificial neural networks—such networks act like small artificial brains and provide the benefit of flexibility in application.
b) Bayesian networks—these networks are suitable for learning relationships between different features, and for using domain knowledge of these relationships.
c) Bayesian kernel methods (such as Gaussian Processes)—can quantify the uncertainty of predictions made and can identify what training examples are required to improve such uncertainty.
d) reinforcement learning—which is a field of machine learning where a policy (such as what treatment to recommend to someone with a given sleep disorder) is improved over time by trial and error. This type of machine learning would for example allow treatment recommendations to be improved over time as the system learns more about what people like, and can be made to understand and predict treatment preferences for different categories of users.

It will be understood to persons skilled in the art of the invention that modifications may be made without departing from the spirit and scope of the invention. The embodiments and/or examples as described herein are therefore to be considered as illustrative and not restrictive.

1. A computer-implemented method for cluster-based recommendation generation regarding sleep disorders, the method comprising:
a server system transmitting query program code to a client device, wherein the query program code is executable by the client device to cause the client device to transmit one or more response objects encoding a response and further responses to the server system;
the server system receiving the one or more response objects from the client device and determining the response and further responses encoded in the one or more response objects;
a clustering module of the server system identifying one or more clusters of sleep disorder user data that most closely relate to the determined response and determined further responses;
a recommendation module of the server system identifying a sleep disorder based on the determined response, the determined further responses and the identified one or more clusters;
the recommendation module generating one or more recommendations based on the identified sleep disorder, the determined response, the determined further responses and the identified one or more clusters; and
the server system encoding the generated one or more recommendations in a recommendations object and making the recommendations object accessible to the client device.

2. The method of claim 1, wherein:
the query program code comprises a query object and one or more further query objects;
the response is a response to the query object;
the further responses are responses to a subset of the one or more further query objects; and
the subset of the one or more further query objects is determined based on the response object.

3. The method of claim 2, wherein the determining of the subset of the one or more further query objects is performed by the server system.

4. The method of claim 2, wherein the determining of the subset of the one or more further query objects is performed by the client device.

5. The method of claim 1, wherein at least one of the one or more further query objects is directed to a sensor and at least one of the one or more response objects comprises health data from the sensor.

6. The method of claim 5, wherein the health data comprises one or more of: cardiac activity measurement data, physical activity measurement data, blood pressure measurement data, respiratory activity measurement data, heart rate data, movement data, breathing sound data or breathing rate data.

7. The method of claim 1, wherein the recommendation module further identifies one or more secondary sleep disorders based on the determined response, the determined further responses and the identified one or more clusters.

8. The method of claim 1, wherein the clustering module performs clustering based on any one of: similarity learning, distance metrics, feature vector comparison or agglomerative clustering.

9. The method of claim 1, wherein at least one of the one or more response objects comprises free text and a natural language processing module of the server system is configured to process the free text to determine an input to the recommendation module.

10. The method of claim 1, further comprising:
receiving feedback input via the client device regarding the one or more recommendations;
reconfiguring a recommendation model of the recommendation module to take into account the received feedback input.

11. A computer-implemented method for cluster-based sleep disorder related recommendation prioritization, the method comprising:
a server system transmitting query program code to a client device, wherein the query program code is executable by the client device to cause the client device to transmit one or more response objects encoding a response and further responses to the server system;
the server system receiving the one or more response objects from the client device and determining the response and further responses encoded in the one or more response objects;
a clustering module of the server system identifying one or more clusters of sleep disorder user data that most closely relate to the determined response and determined further responses;
a recommendation module of the server system identifying a sleep disorder based on the determined response, determined further responses and the identified one or more clusters;
the recommendation module generating one or more recommendations based on the identified sleep disorder, the determined response, the determined further responses and the identified one or more clusters; and
determining a priority of each of the one or more recommendations based on the identified one or more clusters of sleep disorder user data;
the server system encoding the generated one or more prioritized recommendations in a recommendations object and making the recommendations object accessible to the client device.

12. The method of claim 11, wherein:
the query program code comprises a query object and one or more further query objects;
the response is a response to the query object;
the further responses are responses to a subset of the one or more further query objects; and
the subset of the one or more further query objects is determined based on the response.

13. The method of claim 12, wherein the determining of the subset of the one or more further query objects is performed by the server system.

14. The method of claim 12, wherein the determining of the subset of the one or more further query objects is performed by the client device.

15. The method of claim 11, wherein at least one of the one or more further query objects is directed to a sensor and at least one of the one or more response objects comprises health data from the sensor.

16. The method of claim 15, wherein the health data comprises one or more of: cardiac activity measurement data, physical activity measurement data, blood pressure measurement data, respiratory activity measurement data, heart rate data, movement data, breathing sound data or breathing rate data.

17. The method of claim 11,
wherein the identified sleep disorder is a primary sleep disorder; and
wherein the recommendation module further identifies one or more secondary sleep disorders based on the determined response, the determined further responses and the identified one or more clusters.

18. The method of claim 11, wherein the clustering module performs clustering based on any one of: similarity learning, distance metrics, feature vector comparison or agglomerative clustering.

19. The method of claim 11, wherein at least one of the one or more response objects comprises free text and a natural language processing module of the server system is configured to process the free text to determine an input to the recommendation module.

20. The method of claim 11, further comprising:
receiving feedback input via the client device regarding the one or more recommendations;
reconfiguring a recommendation model of the recommendation module to take into account the received feedback input.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)
